(19) United States
(12) Patent Application Publication
(10) Pub. No.: US 2026/0044715 A1
(43) Pub. Date: Feb. 12, 2026

(54) FAULT MONITORING AND DIAGNOSIS METHOD FOR GRANULATION PROCESS BASED ON VARIATIONAL AUTOENCODER AND CONTRIBUTION GRAPH

(71) Applicant: Northeastern University at Qinhuangdao, Qinhuangdao (CN)

(72) Inventors: Zhengsong WANG, Qinhuangdao (CN); Xiaochen LI, Qinhuangdao (CN); Yanqiu YANG, Qinhuangdao (CN); Le YANG, Qinhuangdao (CN)

(73) Assignee: Northeastern University at Qinhuangdao, Qinhuangdao (CN)

(21) Appl. No.: 19/214,106

(22) Filed: May 21, 2025

(30) Foreign Application Priority Data
Aug. 8, 2024 (CN) 202411085834.7

(51) Int. Cl. G06N 3/0455; G16H 20/10; G16H 40/60

(52) U.S. Cl. CPC G06N 3/0455; G16H 20/10; G16H 40/60

(57) ABSTRACT

A monitoring and fault diagnosis method for a continuous granulation process of drug particles based on a variational autoencoder and a contribution graph is provided. By constructing and training the variational autoencoder model, this method achieves dimensionality reduction and feature extraction of high-dimensional data, and generates latent vectors through reparameterization tricks to reconstruct the input data. This method designs monitoring statistics based on KL divergence for real-time monitoring of critical quality attributes and critical process parameters in the production process. Additionally, the contribution graph method is used to evaluate the effect of each variable on the monitoring statistics, so as to realize the accurate diagnosis and location of the fault. The monitoring and fault diagnosis method has strong adaptability and high monitoring accuracy, which can detect potential faults early, avoid the accumulation of quality problems, improve production efficiency, reduce production costs, and ensure drug quality and patient safety.

### CROSS REFERENCE TO THE RELATED APPLICATIONS

This application is based upon and claims priority to Chinese Patent Application No. 202411085834.7, filed on Aug. 8, 2024, the entire contents of which are incorporated herein by reference.

### TECHNICAL FIELD

The present invention belongs to the field of raw material mixing and continuous granulation technology, and relates to the monitoring and fault diagnosis of critical quality attributes of drug particles, specifically a monitoring and fault diagnosis method for continuous granulation process of drug particles based on variational autoencoder and contribution graph method.

### BACKGROUND

With the promotion of Pharma 4.0 (the application of Industry 4.0 in the pharmaceutical field) in the past decade, the continuous pharmaceutical model has gradually become dominant in the pharmaceutical industry due to its ability to increase drug production efficiency, reduce drug manufacturing costs, improve drug product quality, and enhance the flexibility of production and other strengths.

The quality of pharmaceuticals must be monitored strictly to ensure that each batch meets standards, as it is directly related to the health and safety of patients. Due to the complexity and high efficiency of the continuous pharmaceutical process, precise control of each production step is required. Real-time monitoring of critical quality attributes and critical process parameters can identify anomalies in a timely manner, rapidly diagnose faults, and avoid the accumulation of quality problems and production interruptions, thus improving production efficiency and reducing costs. This is of great significance in ensuring drug quality and patient safety.

With the rapid development of automation and informatization of industrial processes, a large amount of data has been collected and stored in industrial production, and data-driven process monitoring technology has developed rapidly accordingly. In the field of continuous pharmaceutical process monitoring, one of the most popular monitoring technologies is multivariate statistical process monitoring (MSPM), such as principal component analysis (PCA), partial least squares (PLS), and so on.

Nevertheless, modern industrial production processes are becoming more and more complex, with a high degree of correlation and nonlinearity between the process variables. The conventional MSPM methods are difficult to deal with this situation, which then affects their monitoring performance. In order to solve the problem of monitoring nonlinear processes, researchers and scholars have proposed different types of monitoring methods. Wherein, the most commonly used methods are kernel methods, such as kernel principal component analysis (KPCA), kernel partial least squares (KPLS), and so on. However, due to the limitation of kernel technology, these methods are only applicable to the processing of small and medium samples. In order to further improve the monitoring performance, some scholars apply deep learning methods to the process monitoring field, such as autoencoder (AE), variational autoencoder (VAE), restricted Boltzmann machine (RBM), and so on.

However, none of the above networks can handle the non-Gaussianity of data well. The nonlinearity and non-Gaussianity of data directly affect the extraction of data features. The nonlinear and non-Gaussianity of the data can be processed to extract the essential features of the data.

### SUMMARY

In order to solve the problems existing in the existing technology, the present invention provides a monitoring and fault diagnosis method for a continuous granulation process of drug particles based on a variational autoencoder and a contribution graph method. The present invention is not limited by prior knowledge, can capture complex relationships and nonlinear characteristics between variables in the data, adaptively extract feature information hidden in original data, map high-dimensional data to low-dimensional latent space, realize a dimensionality reduction and a feature extraction of the data, and has strong adaptability.

The technical scheme of the present invention is:
a monitoring and fault diagnosis method for a continuous granulation process of drug particles based on a variational autoencoder and a contribution graph method, including the following steps:
step 1: collection and preprocessing of historical data:
simulating an actual production process by using an established wet granulation model of a feeding blender and a twin screw granulation, collecting attributes and operating variables such as a mass flow rate of raw materials at an inlet of the feeding blender, the mass flow rate of an excipient, a rotational speed of the feeding blender, the mass flow rate of an outlet of the feeding blender, the mass flow rate of an adhesive, a screw rotational speed of the twin screw granulation, an average residence time of particles in equipment, a particle size and the moisture content of particles during a normal operation.

Standardizing the collected historical production data of the granulation process as a training set by using the following formula.

$${\tilde{x}}_{j k} = ( {x}_{j k} - {\overline{x}}_{k} ) / {σ}_{k}$$

where xⱼₖ is a j^{th} data of a k^{th} variable in the data set, x̅ₖ is a mean of the k^{th} variable in the data set, and σk is a standard deviation of the k^{th} variable in the data set.

Step 2: establishing a variational autoencoder model:
the variational autoencoder model consists of a probabilistic encoder and a probabilistic decoder.

The encoder module consists of three fully connected layers, with an input dimension of 9, after being processed by a ReLU activation function, output dimensions are 128 and 64 respectively, finally, outputting the mean and logarithmic variance of the latent space; generating a latent vector z by reparameterization tricks. The decoder module also consists of three fully connected layers, with the input dimension of 3, after being processed by the ReLU activation function, the output dimensions are 64 and 128 respectively, and a final output dimension is 9, using a Sigmoid activation function to decode the latent space vector into high-dimensional input data form and reconstruct the original input data.

Step 3: training the variational autoencoder model established in step 2:
during a training process, optimization steps of the model include a forward propagation, a loss calculation, and a parameter update. During the forward propagation, generating the mean and logarithmic variance of the input data by an encoder, obtaining the latent vector by the reparameterization tricks, and then generating reconstructed data by a decoder. The loss function consists of a reconstruction loss, and a Kullback-Leibler (KL) divergence loss, wherein the reconstruction loss measures a difference between the reconstructed data and the original input data, and the KL divergence loss measures the difference between a latent spatial distribution and a standard normal distribution. Finally, performing a backpropagation according to a total loss, updating the parameters of the encoder and decoder.

Step 4: designing a monitoring statistic and calculating a control limit:
regarding the distribution generated in the latent space of data when operating normally as a baseline distribution. Regarding the KL divergence between the distribution of monitoring data in the latent space and the baseline distribution as the monitoring statistic, the monitoring statistic is used to measure a degree of deviation of monitoring data from normal operating conditions, and is recorded as a Kullback-Leibler divergence (KLD).

In a process of calculating the control limit, firstly, inputting a K set of normal operation data into the variational autoencoder model, and calculating the KLD between it and the baseline distribution, and recording. Secondly, estimating a probability density function of this set of the KLD by using a kernel density estimation method, and calculating the control limit at a confidence level of 0.98.

Step 5: performing a manufacturing process monitoring.

Step 6: calculating a contribution degree of each variable to the monitoring statistics, and then realizing the fault diagnosis.

In order to evaluate the effect of each variable on the monitoring statistic KLD, the present invention uses the following methods for processing.

First, there is a historical normal data set X:

$$X = {\{ {x}_{1}^{( i )} , {x}_{2}^{( i )} , … , {x}_{n}^{( i )} \}}_{n \times i}^{T} , i = 1 , 2 , … , I$$

where

$${x}_{n}^{( i )}$$

denotes an n^{th} data corresponding to an i^{th} variable in a normal data set.

Secondly, a monitored data set X′ with anomalies:

$${X}^{′} = {\{ {x}_{1}^{′ ( i )} , {x}_{2}^{′ ( i )} , … , {x}_{p}^{′ ( i )} \}}_{p \times i}^{T} , i = 1 , 2 , … , I$$

where

$${x}_{p}^{′ ( i )}$$

is an n^{th} data corresponding to a p^{th} variable in an abnormal data set.

In order to calculate the effect of each variable on the monitoring statistic KLD, the data are processed as follows:

$${X}^{D ( i )} = [ \begin{matrix}{X}_{1}^{D ( i )} \\ {X}_{2}^{D ( i )} \\ {X}_{3}^{D ( i )} \\ … \\ {X}_{p}^{D ( i )}\end{matrix} ] = \{ \begin{matrix}{x}_{1}^{( 1 )} & … & {x}_{1}^{′ ( i )} & … & {x}_{1}^{( I )} \\ {x}_{2}^{( 1 )} & … & {x}_{2}^{′ ( i )} & … & {x}_{2}^{( I )} \\ {x}_{3}^{( 1 )} & … & {x}_{3}^{′ ( i )} & … & {x}_{3}^{( I )} \\ … & … & … & … & … \\ {x}_{p}^{( 1 )} & … & {x}_{p}^{′ ( i )} & … & {x}_{p}^{( I )}\end{matrix} \}$$

where X^{D(i)} denotes an i^{th} variable in the abnormal data is replaced by the i^{th} variable in the normal historical data, and p denotes that the production process is sampled p times.

The replaced I set of X^{D(i)} are respectively sent to the variational autoencoder monitoring model to calculate the KL divergence between it and the baseline distribution, and is recorded as KLD⁽ⁱ⁾.

Therefore, the contribution degree R of each variable to KLD can be calculated by the following formula:

$$R = \frac{{\sum }_{p} {KLD}^{( i )}}{{\sum }_{i} {\sum }_{p} K L {D}^{( i )}} \times 100 \%$$

Through the above method, the contribution degree of each variable to KLD can be analyzed, and a fault location can be located more accurately.

The beneficial effects of the present invention are: the present invention achieves high-accuracy monitoring and fault diagnosis of the production process of drug particles by introducing the variational autoencoder combined with the contribution graph method. The method adaptively extracts complex nonlinear features hidden in the original data and maps the high-dimensional data to a low-dimensional latent space, which improves the monitoring accuracy and fault diagnosis capability. Compared with the conventional method, the present invention can find and locate potential faults earlier, avoid the accumulation of quality problems, have strong adaptability to different production environments and process conditions, improve production efficiency and reduce production costs. Meanwhile, the present invention is an advanced and effective method for monitoring and fault diagnosis in the production process of drug particles, as it performs excellently in handling the non-Gaussianity of data, and can extract the intrinsic characteristics of the data, so as to guarantee the quality of the drug and the safety of the patients.

### BRIEF DESCRIPTION OF THE DRAWINGS

FIG. 1 is a schematic diagram of a variational autoencoder model;

FIG. 2 is a flow chart of a method of the present invention;

FIG. 3 is a calculation process diagram of a control limit;

FIG. 4 is a diagram of a normal data distribution and fault monitoring results;

FIG. 5 is a comparison diagram of fault diagnosis results between a method of the present invention and a conventional PCA method;

FIG. 6 is a comparison diagram of monitoring performance indicators between a method of the present invention and a conventional PCA method.

### DETAILED DESCRIPTION OF THE EMBODIMENTS

The following is a detailed description of specific embodiments of the present invention in the context of the technical scheme and the accompanying drawings.

In the embodiment, nine critical variables in the process of particle production were selected for monitoring and fault diagnosis. The flow chart of the drug particle quality monitoring method in this process is shown in FIG. 2, including the following steps:
step 1: collection and preprocessing of historical data:
the actual production process is simulated by using the established wet granulation model of the feeding blender and the twin screw granulation, attributes and operating variables such as the mass flow rate of raw materials at the inlet of the feeding blender, the mass flow rate of the excipient, the rotational speed of the feeding blender, the mass flow rate of the outlet of the feeding blender, the mass flow rate of the adhesive, the screw rotational speed of the twin screw granulation, the average residence time of particles in equipment, the particle size and the moisture content of particles are collected during the normal operation.

The collected historical production data of the granulation process is standardized as the training set by using the following formula.

$${\tilde{x}}_{j k} = ( {x}_{j k} - {\overline{x}}_{k} ) / {σ}_{k}$$

where xⱼₖ is the j^{th} data of the k^{th} variable in the data set, x̅ₖ is the mean of the k^{th} variable in the data set, and σₖ is the standard deviation of the k^{th} variable in the data set.

Step 2: the variational autoencoder model is established:

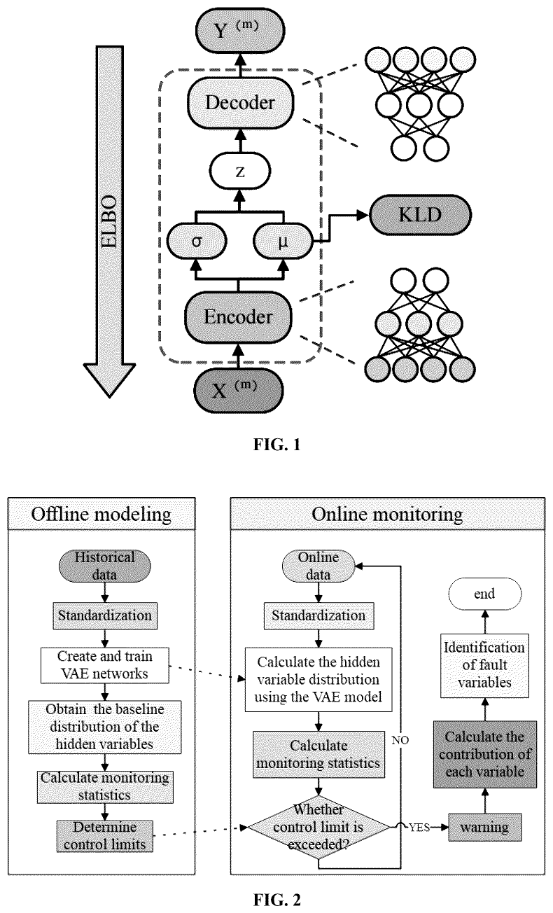

FIG. 1 shows the flow of data in the variational autoencoder. The encoder module consists of three fully connected layers, with the input dimension of 9, after being processed by the ReLU activation function, output dimensions are 128 and 64 respectively, finally, the mean and logarithmic variance of the latent space are output; the latent vector z is generated by reparameterization tricks. The decoder module also consists of three fully connected layers, with the input dimension of 3, after being processed by the ReLU activation function, the output dimensions are 64 and 128 respectively, and the final output dimension is 9, the Sigmoid activation function is used to decode the latent space vector into high-dimensional input data form and reconstruct the original input data.

Step 3: the variational autoencoder model established in step 2 is trained:
during the training process, the optimization steps of the model include the forward propagation, the loss calculation, and the parameter update. During the forward propagation, generating the mean and logarithmic variance of the input data by the encoder, the latent vector is obtained by the reparameterization tricks, and then the reconstructed data is generated by the decoder. The loss function consists of the reconstruction loss and the KL divergence loss, wherein the reconstruction loss measures the difference between the reconstructed data and the original input data, and the KL divergence loss measures the difference between the latent spatial distribution and the standard normal distribution. Finally, the backpropagation is performed according to the total loss, and the parameters of the encoder and decoder are updated.

Step 4: the monitoring statistic is designed and the control limit is calculated:
the distribution generated in the latent space of data when operating normally is regarded as the baseline distribution. The KL divergence between the distribution of monitoring data in the latent space and the baseline distribution is regarded as the monitoring statistic, the monitoring statistic is used to measure the degree of deviation of monitoring data from normal operating conditions, and is recorded as the KLD.

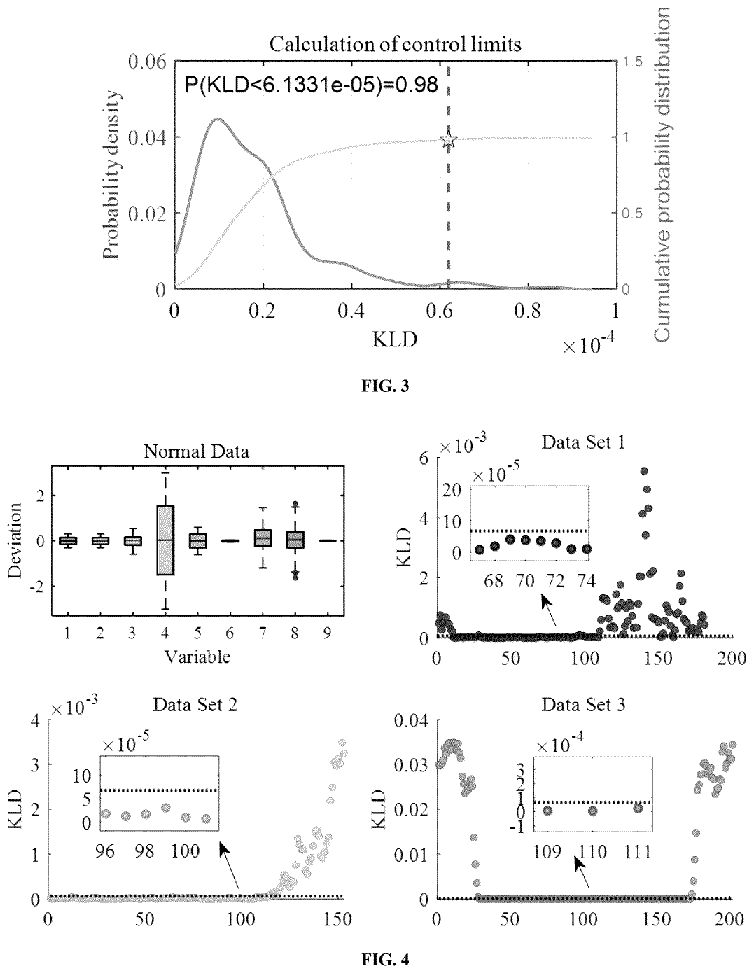

FIG. 3 is the control limit specific kernel density estimation results. In the process of calculating the control limit, firstly, the K set of normal operation data is input into the variational autoencoder model, and the KLD between it and the baseline distribution is calculated, and recorded. Secondly, the probability density function of this set of the KLD is estimated by using the kernel density estimation method, and the control limit at the confidence level of 0.98 is calculated.

Step 5: the manufacturing process monitoring is performed. FIG. 4 shows the normal data distribution and the monitoring results of the three types of faults designed in Table 1. Taking the operating condition shown in Table 1 as an example, the monitoring values of each variable fluctuate around the baseline value, and the fluctuation range does not exceed 5%. The remaining three diagrams show the performance of the process monitoring method based on variational autoencoder when confronting of 1 to 3 types of faults. It can be observed that when these three types of faults occur, the value of the monitoring statistic KLD increases rapidly and is significantly higher than the control limit. It can be concluded that the monitoring method based on the variational autoencoder can monitor the occurrence of the types of faults such as oscillations, steps, and slow drifts in a timely manner, which proves the effectiveness of the monitoring method.

TABLE 1
Example fault design

| | Fault variable | Fault type |
|---|---|---|
| Data set 1 | Water | Oscillation |
| Data set 2 | API | Step |
| Data set 3 | Excipient | Slow drift |

Step 6: the contribution degree of each variable to the monitoring statistics is calculated, and then the fault diagnosis is realized.

In order to evaluate the effect of each variable on the monitoring statistic KLD, the present invention uses the following methods for processing.

First, there is the historical normal data set X:

$$X = {\{ {x}_{1}^{( i )} , {x}_{2}^{( i )} , … , {x}_{n}^{( i )} \}}_{n \times i}^{T} , i = 1 , 2 , … , 9$$

where

$${x}_{n}^{( i )}$$

denotes the n^{th} data corresponding to the i^{th} variable in the normal data set.

Secondly, the monitored data set X′ with anomalies:

$${X}^{′} = {\{ {x}_{1}^{′ ( i )} , {x}_{2}^{′ ( i )} , … , {x}_{n}^{′ ( i )} \}}_{p \times i}^{T} , i = 1 , 2 , … , 9$$

where

$${x}_{p}^{′ ( i )}$$

is the n^{th} data corresponding to the p^{th} variable in the abnormal data set.

In order to calculate the effect of each variable on the monitoring statistic KLD, the data are processed as follows:

$${X}^{D ( i )} = [ \begin{matrix}{X}_{1}^{D ( i )} \\ {X}_{2}^{D ( i )} \\ {X}_{3}^{D ( i )} \\ … \\ {X}_{p}^{D ( i )}\end{matrix} ] = \{ \begin{matrix}{x}_{1}^{( 1 )} & … & {x}_{1}^{′ ( i )} & … & {x}_{1}^{( 9 )} \\ {x}_{2}^{( 1 )} & … & {x}_{2}^{′ ( i )} & … & {x}_{2}^{( 9 )} \\ {x}_{3}^{( 1 )} & … & {x}_{3}^{′ ( i )} & … & {x}_{3}^{( 9 )} \\ … & … & … & … & … \\ {x}_{p}^{( 1 )} & … & {x}_{p}^{′ ( i )} & … & {x}_{p}^{( I )}\end{matrix} \}$$

where X^{D(i)} denotes the i^{th} variable in the abnormal data is replaced by the i^{th} variable in the normal historical data, and p denotes that the production process is sampled p times.

The replaced 9 sets of X^{D(i)} are respectively sent to the variational autoencoder monitoring model to calculate the KL divergence between it and the baseline distribution, and is recorded as KLD⁽ⁱ⁾.

Therefore, the contribution degree R of each variable to KLD can be calculated by the following formula:

$$R = \frac{{\sum }_{p} {KLD}^{( i )}}{{\sum }_{i} {\sum }_{p} K L {D}^{( i )}} \times 100 \%$$

Through the above method, the contribution degree of each variable to KLD can be analyzed, and the fault location can be located more accurately.

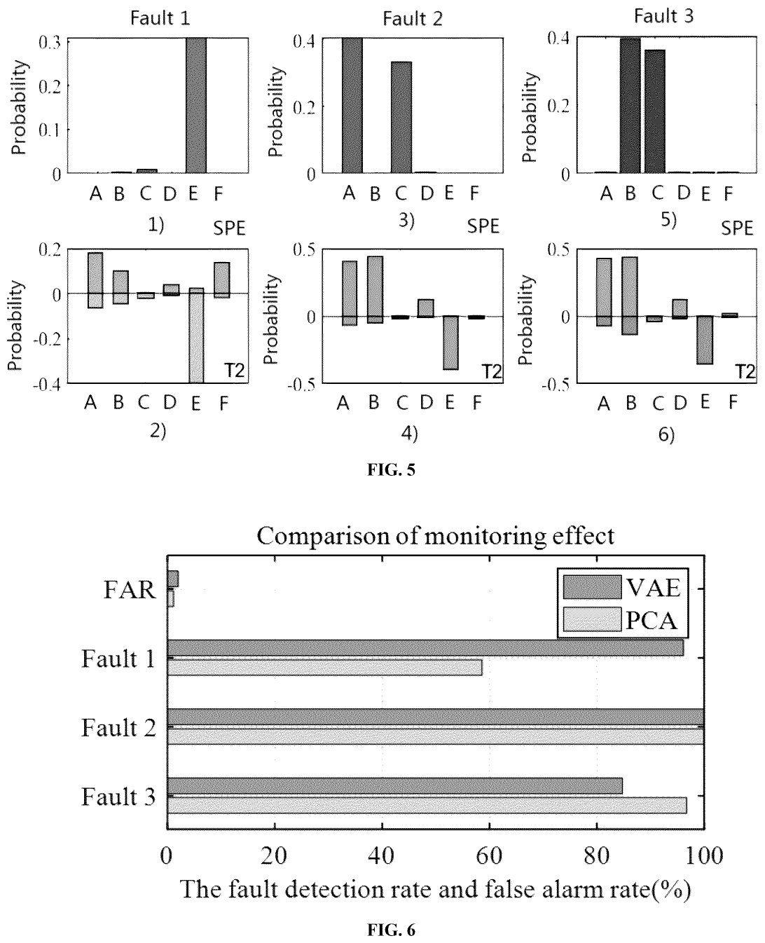

FIG. 5 shows the comparison results of fault diagnosis results between the method of the present invention and the conventional PCA method. In FIG. 5, 1), 3), and 5) denote the results of fault variable identification based on the variational autoencoder fault graph method, and 2), 4), and 6) denote the results of fault identification based on PCA.

It can be seen that the T2 statistic in PCA has a good effect on the fault identification of oscillation type, but it can not identify the fault of step and drift type. However, the SPE statistic is not ideal for the identification of three types of faults, the non-fault variables are greatly polluted by the fault variables, and the fault location cannot be accurately identified. In contrast, the monitoring method based on variational autoencoder can better identify the location of three types of faults, such as oscillation, step and drift. Based on the experimental results, it can be preliminarily concluded that the monitoring method based on the variational autoencoder is superior to the conventional PCA-based monitoring method.

It is required to analyze the monitoring results to compare the effect of the two monitoring methods in a deeper manner. In process monitoring, false alarm rate (FAR) and abnormal detection rate (ADR) are the main indicators to evaluate the effect of monitoring methods, which are collectively referred to as monitoring performance indicators.

The calculation methods of ADR and FAR are as follows:

$$\begin{matrix}ADR = \frac{{N}_{T a}}{{N}_{a}} \times 100 \% \\ FAR = \frac{{N}_{F a}}{{N}_{n}} \times 100 \%\end{matrix}$$

where N_{Ta} is the number of detected true abnormal samples, N_{Fa} is the number of detected false abnormal samples, Nₐ is the number of abnormal samples in the data set, and Nₙ is the number of normal samples in the data set.

The above collected data sets are still used to calculate the FAR of the two monitoring methods during normal operation as well as the fault detection rates of the two monitoring methods during the occurrence of faults 1-3, respectively, and the results are shown in the FIG. 6.

It can be seen from FIG. 6 that the FAR of the two monitoring methods remains at a very low level when dealing with normal data, which will not affect normal production. However, when dealing with different types of fault data, the monitoring method based on the variational autoencoder model performs well. For three types of faults such as oscillation, step and slow drift, the ADR remains above 85%, which shows a high performance level. In contrast, when the PCA-based method confronts oscillation faults, the ADR is less than 60%, and the monitoring task cannot be effectively completed.

Therefore, it can be concluded that the variational autoencoder has greater advantages than PCA in both process monitoring and fault diagnosis, and can effectively complete the corresponding monitoring and diagnosis tasks.

What is claimed is:

1. A monitoring and fault diagnosis method for a continuous granulation process of drug particles based on a variational autoencoder and a contribution graph method, comprising the following steps:
step 1: collection and preprocessing of historical data:
simulating an actual production process by using an established wet granulation model of a feeding blender and a twin screw granulation, collecting critical raw material attributes and operating variables during a normal operation, and standardizing collected historical production data of the continuous granulation process as a training set;
step 2: establishing a variational autoencoder model:
step 3: training the variational autoencoder model established in step 2:
during a training process, optimization steps of the variational autoencoder model comprise a forward propagation, a loss calculation, and a parameter update; during the forward propagation, generating a mean and logarithmic variance of input data by an encoder, obtaining a latent vector by reparameterization tricks, and then generating reconstructed data by a decoder; a loss function comprises a reconstruction loss, and a Kullback-Leibler (KL) divergence loss, wherein the reconstruction loss measures a difference between the reconstructed data and original input data, and the KL divergence loss measures a difference between a latent spatial distribution and a standard normal distribution; finally, performing a backpropagation according to a total loss, and updating parameters of the encoder and the decoder;
step 4: designing a monitoring statistic and calculating a control limit:
regarding a distribution generated in a latent space of data when operating normally as a baseline distribution; regarding a KL divergence between a distribution of monitoring data in the latent space and the baseline distribution as the monitoring statistic, wherein the monitoring statistic is used to measure a degree of deviation of the monitoring data from normal operating conditions, and is recorded as a Kullback-Leibler divergence (KLD); in a process of calculating the control limit, firstly, inputting a K set of normal operation data into the variational autoencoder model, and calculating the KLD between the K set of normal operation data and the baseline distribution, and recording; secondly, estimating a probability density function of this set of the KLD by using a kernel density estimation method, and calculating the control limit at a confidence level of 0.98;
step 5: performing a manufacturing process monitoring; and
step 6: calculating a contribution degree of each variable to the monitoring statistic, and then realizing fault diagnosis;
wherein in order to calculate an effect of each variable on the monitoring statistic KLD, data are processed as follows:
$${X}^{D ( i )} = [ \begin{matrix}{X}_{1}^{D ( i )} \\ {X}_{2}^{D ( i )} \\ {X}_{3}^{D ( i )} \\ … \\ {X}_{p}^{D ( i )}\end{matrix} ] = \{ \begin{matrix}{x}_{1}^{( 1 )} & … & {x}_{1}^{′ ( i )} & … & {x}_{1}^{( I )} \\ {x}_{2}^{( 1 )} & … & {x}_{2}^{′ ( i )} & … & {x}_{2}^{( I )} \\ {x}_{3}^{( 1 )} & … & {x}_{3}^{′ ( i )} & … & {x}_{3}^{( I )} \\ … & … & … & … & … \\ {x}_{p}^{( 1 )} & … & {x}_{p}^{′ ( i )} & … & {x}_{p}^{( I )}\end{matrix} \}$$
wherein X^{D(i)} denotes an i^{th} variable in abnormal data is replaced by an i^{th} variable in normal historical data, and p denotes that a production process is sampled for p times,
$${x}_{n}^{( i )}$$
denotes an n^{th} data corresponding to an i^{th} variable in a normal data set, and
$${x}_{p}^{′ ( i )}$$
is an n^{th} data corresponding to a p^{th} variable in an abnormal data set;
the replaced I set of X^{D(i)} are respectively sent to the variational autoencoder monitoring model to calculate the KL divergence between the replaced I set of X^{D(i)} and the baseline distribution, and is recorded as KLD⁽ⁱ⁾,
therefore, the contribution degree R of each variable to KLD is calculated by the following formula:
$$R = \frac{{\sum }_{p} {KLD}^{( i )}}{{\sum }_{i} {\sum }_{p} K L {D}^{( i )}} \times 100 \%$$
through the above method, the effect of each variable to KLD is analyzed, and a fault location is located more accurately.

2. The monitoring and fault diagnosis method according to claim 1, wherein an encoder module comprises three fully connected layers, with an input dimension of 9, after being processed by a ReLU activation function, output dimensions are 128 and 64 respectively, finally, the mean and logarithmic variance of the latent space are output; a latent vector z is generated by the reparameterization tricks; a decoder module also comprises three fully connected layers, with the input dimension of 3, after being processed by the ReLU activation function, the output dimensions are 64 and 128 respectively, and a final output dimension is 9, a Sigmoid activation function is used to decode a latent space vector into high-dimensional input data form and reconstruct the original input data.

3. The monitoring and fault diagnosis method according to claim 1, wherein critical attributes and operating variables in step 1 comprise a mass flow rate of raw materials at an inlet of the feeding blender, a mass flow rate of an excipient, a rotational speed of the feeding blender, a mass flow rate of an outlet of the feeding blender, a mass flow rate of an adhesive, a screw rotational speed of the twin screw granulation, an average residence time of particles in equipment, a particle size and a moisture content of particles.

4. The monitoring and fault diagnosis method according to claim 1, wherein in step 6, by replacing the i^{th} variable in the abnormal data with the i^{th} variable in the normal historical data, the KL divergence between the replaced data and the baseline distribution is calculated to evaluate the effect of each variable on the monitoring statistic KLD.
